(19) United States
(12) Patent Application Publication
(10) Pub. No.: US 2009/0287189 A1
(43) Pub. Date: Nov. 19, 2009

(54) OPTIMAL RADIOPAQUE CATHETER

(75) Inventor: Wan Suwito, Sandy, UT (US)

Correspondence Address: David W. Highet, VP & Chief IP Counsel;Becton, Dickinson and Company, (Kirton & McConkie), 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 12/120,436

(22) Filed: May 14, 2008

(51) Int. Cl. A61M 25/098; A61M 25/18

(52) U.S. Cl. 604/529; 604/533

(57) ABSTRACT

A vascular catheter embedded with a radiopaque material providing a distinct, non-physiological pattern that may be easily detected on a radiograph. The radiopaque material may be embedded within the wall of the catheter in an open wound, helical formation. Detection of the catheter by x-ray is increased due to the non-physiological radiograph pattern and due to the increased presence of the radiopaque material. The non-physiological formation increases the radiopacity of the catheter, yet requires less radiopaque material than traditional radiopaque catheters. The non-physiological formation and decreased amount of radiopaque material provides for a more detectible and less rigid catheter that is resistant to kinks and occlusions.

### BACKGROUND OF THE INVENTION

The present disclosure relates generally to vascular access devices and methods, including catheter assemblies and devices used with catheter assemblies. Generally, vascular access devices are used for communicating fluid with the vascular system of patients. For example, catheters are used for infusing fluid, such as saline solution, various medicaments, or total parenteral nutrition, into a patient. Catheters are also used for withdrawing blood from a patient or monitoring various parameters of the patient's vascular system.

Generally a catheter comprises a tubular member having one end attached to a catheter adapter. The catheter may be inserted into a patient via an introducer needle or a surgical procedure. Following insertion of the catheter, the catheter adapter remains uninserted and is secured to the patient via an adhesive strip or bandaging material. The attachment of the catheter to the catheter adapter immobilizes the catheter and prevents the catheter from being released into the venous system of the patient. However, there are cases where a catheter has become detached from the catheter adapter and floated downstream within the venous system of the patient.

A catheter may become detached from the catheter adapter either due to a malfunction of the catheter assembly or by human error. A catheter assembly may malfunction where the means for coupling the catheter to the catheter adapter fails. For example, where the catheter is coupled to the catheter adapter via a mechanical fastener, the mechanical fastener may fail thereby releasing the catheter into the venous system of the patient. Human error, such as inadvertently severing the catheter, may also occur thereby releasing the catheter into the venous system of the patient. Once released into the venous system of the patient, the catheter must be located, immobilized and removed. Recovering the released catheter must be done quickly to avoid any secondary complications, such as a blocked artery or an embolism, which may occur due to the detached catheter. Catheters are generally made from a clear polymer, such as polyurethane. Once released into the venous system of a patient, the catheter visually blends into the surrounding tissue making the catheter difficult to detect.

Since the base catheter material is highly transmissible to X-rays, methods have been developed to make catheters absorb more X-rays. For example, a radiopaque material may be added to the base material of the catheter. Radiopaque material adsorbs X-rays thereby limiting the X-rays that reach the x-ray film. As such, the negative resultant radiograph displays dark shades for high transmissible areas, light shades for low transmissible areas, and in between shades dependent upon the level of X-rays reaching the film. As such, a released catheter may be detected within a patient by x-raying the patient and searching the radiograph for the dark and light patterns of the catheter against the background of the patient's body.

In traditional catheters, the radiopaque material is added either evenly, resulting in uniformly opaque catheters, or is distributed in linear stripes leaving clear areas between the stripes for viewing fluid flow. In either configuration, the resultant radiograph of the opaque catheters yields linear patterns similar to the radiograph patterns of other surrounding tissues. Bones, muscles, ligaments, veins and other tissues are all generally linear and therefore display linear patterns when exposed to x-ray film. Additionally, radiopaque material is generally stiff or semi-rigid. Adding a sufficient amount of radiopaque material to a catheter may decrease the flexibility of the catheter. As such, the catheter becomes more difficult to navigate within a patient and may be more likely to kink or occlude when making necessary flexures.

Therefore, a need exists for a radiopaque catheter that may be easily detected on a radiograph, yet remains flexible and resistant to kinks or occlusions. Additionally, an improved radiopaque catheter is needed that provides a window for observing the flow of a fluid through the catheter. Accordingly, the present disclosure presents systems and methods to provide such optimized radiopaque catheters to resolve the previously discussed issues.

### BRIEF SUMMARY OF THE INVENTION

The systems and methods of the present disclosure have been developed in response to problems and needs in the art that have not yet been fully resolved by currently available radiopaque catheters and methods. Thus, these systems and methods are developed to provide for safer and more efficient infusion procedures.

One aspect of the present disclosure provides a catheter assembly comprising a catheter adapter and a catheter. The catheter adapter generally comprises a tubular body coupled to a first end of the catheter. A lumen of the catheter adapter may be in fluid communication with a lumen of the catheter such that a fluid may be infused from the catheter adapter, through the catheter and into a patient. The catheter adapter may further include a valve or vent for controlling the flow of an infusant through the catheter assembly. The catheter adapter may also include a flashback chamber for visually confirming proper insertion of the catheter.

The catheter adapter may include a needle port for receiving an introducer needle. A tip of the introducer needle may also extend beyond a tip of the catheter such that the tip of the introducer needle may be used to provide an opening in a patient's skin for inserting the catheter. The catheter adapter may also include other features such as an access port and catheter wings. The access port may be in fluid communication with the lumen of the catheter adapter and may be coupled to an infusant source, such as an intravenous fluid bag. The catheter wings may comprise a semi-flexible material and may be used to grasp the catheter adapter during removal of the catheter.

An end of the catheter adapter may be further modified to couple to another component of the infusion system. The catheter adapter may be modified to couple to a needle shield or a needle hub. Additionally, the catheter adapter may be modified to include an interlocking system for locking the catheter adapter to another component of the infusion system.

The catheter may comprise a length of tubing attached to an end of the catheter adapter. The catheter may comprise a polymer material, such as polyurethane. The catheter may attach to the catheter adapter in a fluidtight manner such that a fluid may be contained within the lumens of the catheter and the catheter adapter. An inner lumen of the catheter may also accommodate the passage of an introducer needle. A tip of the catheter may be tapered to accommodate the insertion of the catheter into an opening in the patient's skin as created by the tip of the introducer needle.

A material may be embedded in a configuration within a wall of the catheter. The material may comprise a radiopaque material and may be embedded within the wall during the extrusion of the catheter. The embedded material may also provide a unique pattern when exposed to x-ray film. For example, the unique pattern may comprise a crisscrossed pattern. The unique pattern may also comprise a non-physiological pattern. Specifically, the pattern may be non-linear and distinct from naturally occurring patterns within the tissues of the patient. The enhanced visibility of the catheter may also be used to track and assist in the placement of the catheter for central access procedures. Additionally, the distinct pattern may be used to quickly locate a severed catheter within a patient whereafter the severed catheter may be immobilized and safely removed from the patient.

The material may be embedded within the wall of the catheter in an open wound, helical formation. The strand diameter and amount of the material may vary depending upon the needs of the catheter. A greater amount of radiopaque material may be used where a less flexible, more visible catheter is desired. A lesser amount of radiopaque material may be used where a more flexible catheter is desired. Various widths, heights, and cross-sectioned shapes of radiopaque material may also be used to affect the overall properties and radiopacity of the catheter.

The embedded material may include one or more strands of radiopaque material. One embodiment of the catheter may include three helical strands of radiopaque material. Each strand may be embedded within the wall of the catheter at a predetermined pitch. The pitch and spacing of the strands may provide a window of clear, unembedded catheter through which the flow of a fluid through the catheter may be observed. The pitch and spacing of the strands may also provide a desired flexibility for the catheter. The pitch and spacing of the strands may also be configured to provide a desired radiopacity for the catheter.

The embedded radiopaque material may also strengthen the wall of the catheter. The strengthened catheter wall may be beneficial for infusion procedures requiring rapid infusion at high pressures. The strengthened catheter wall may also prevent the catheter from becoming kinked or partially occluded.

The catheter may be inserted into a patient in the same manner as traditional catheters. The helical configuration of the radiopaque material may provide a more flexible catheter with increased resistance to kinks and occlusions. The catheter may therefore bend, flex and contour during insertion without kinking or occluding.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is a perspective view of a catheter assembly including a catheter with an embedded material in a helical configuration.

FIG. 2 is a cross section view of the catheter of FIG. 1.

FIG. 3 is a cross section view of the catheter of FIG. 1.

FIG. 4 is a perspective view, shown partially in phantom for clarity, of the catheter of FIGS. 1 and 2 with the tube shown in phantom, and showing cross-sectional walls for clarity.

FIG. 5 is a partially cut away perspective view of a catheter as inserted into a cross-sectioned patient, wherein a radiopaque material is embedded within the wall of the catheter.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

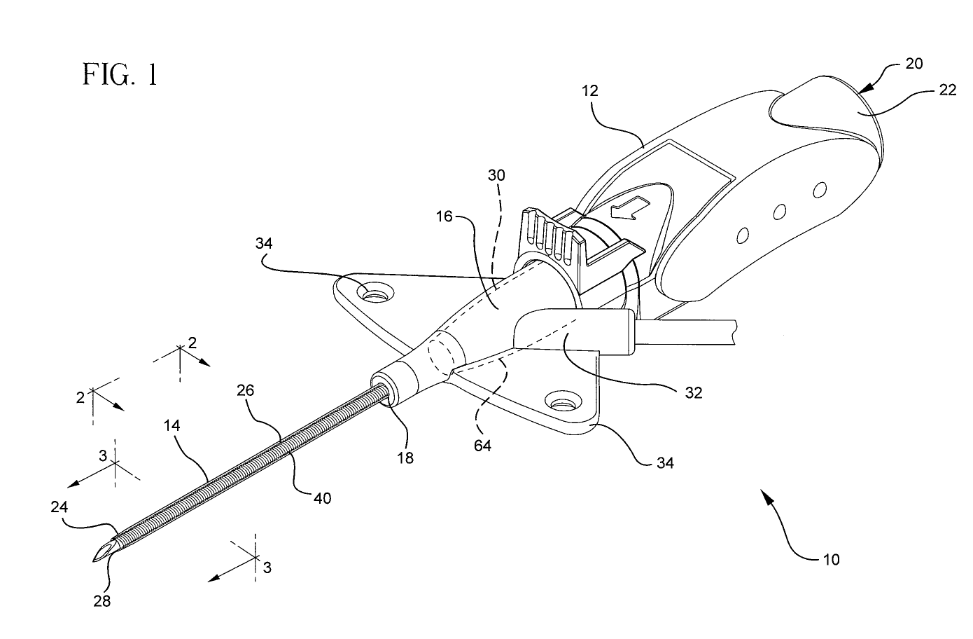

Referring now to FIG. 1, a catheter assembly 10 is illustrated. The catheter assembly 10 comprises a catheter adapter 12 and a catheter 14. The catheter adapter further comprises a generally tubular body 16 coupled to a first end 18 of the catheter 14. As configured, a lumen 30 of the catheter adapter body 16 is in fluid communication with a lumen of the catheter 14. Therefore, a fluid may be infused from the catheter adapter 12, through the catheter 14 and into a vasculature of a patient. Conversely, a fluid may be removed from a patient through the catheter 14. The catheter adapter body 16 may also include a flashback chamber 64 for visually confirming proper insertion of the catheter 14.

The catheter adapter 12 further comprises a needle port 20 located at a first end 22 of the catheter adapter 12. The needle port 20 comprises an opening to the lumen of the catheter adapter 12 configured to receive an introducer needle. An introducer needle may be inserted through the needle port 20 and extend through the lumen 30 of the catheter adapter 12. A tip of the introducer needle may extend beyond the tip 24 of the catheter 14. As such, the introducer needle may provide an opening in a patient's skin for introducing the catheter 14 into the vasculature of the patient.

The catheter adapter 12 may also comprise other features to aid in accessing, positioning, and maneuvering the catheter 14. For example, the catheter adapter 12 may also comprise an access port 32 and catheter wings 34. The access port 32 is in fluid communication with the lumen 30 of the catheter adapter 12 and may be modified to couple an infusant source to the catheter assembly 10. For example, the access port 32 may be attached to an intravenous fluid bag via a length of intravenous tubing. The catheter wings 34 may comprise a semi-flexible material to aid a user in securing the catheter assembly 10 to a patient following insertion of the catheter 14. For example, the catheter wings 34 may provide a broad surface for securing the catheter assembly 10 to a patient via an adhesive strip or a bandaging material. Additionally, the catheter wings 34 may be grasped by a user to remove the catheter 14 from a patient following an infusion therapy.

The first end 22 of the catheter adapter 12 may be further modified to couple to another component of an infusion system. For example, the first end 22 of the catheter adapter 12 may be modified to couple to a needle shield or a needle hub comprising an introducer needle. The first end 22 of the catheter adapter 12 may also be modified to include an interlocking system for locking together the catheter adapter 12 and another component of an infusion system.

The catheter 14 comprises a length of tubing attached to a portion of the catheter adapter body 16. The catheter 14 is generally fabricated from a polymeric material such as nylon, PVC, PVP, silicone, polyurethane and/or polyethylene. The catheter 14 may attach to the catheter adapter body 16 in a fluidtight manner whereby the catheter and the lumen 30 of the catheter adapter 12 are in fluid communication. For example, the catheter 14 may be attached to the catheter adapter body 16 by means of a pressure fitting or an adhesive. The catheter 14 further comprises a lumen 28 with an inner diameter structured to accommodate the insertion of an introducer needle. A tip 24 of the catheter 14 may taper inwardly such that an outer diameter of the catheter 14 is reduced to approximately the outer diameter of an introducer needle. As such, the tapered tip 24 of the catheter 14 facilitates the insertion of the catheter 14 into an opening created by the tip of the introducer needle.

The catheter 14 further comprises a material 40 embedded within a wall 26 of the catheter 14. The material 40 comprises a radiopaque filler embedded within the catheter wall 26 during the manufacturing process of the catheter 14. The radiopaque filler may include any material visible to X-rays. For example, the radiopaque filler may include a chemical salt of bismuth or barium, or an element such as platinum or tungsten. In one embodiment, the radiopaque material is barium sulfate.

As illustrated in FIG. 1, the material 40 is embedded within the wall 26 of the catheter 14 in an open wound, helical formation. The material 40 may be embedded by any method known in the art. For example, the material 40 may be embedded within the wall 26 of the catheter 14 during the extrusion process of the catheter 14. Specifically, a coextrusion process may be used to produce embedded catheters 14.

Plastic tubing, such as a catheter, is manufactured by extruding molten polymer through a die of the desired profile shape. For example, a die may be used to produce various shapes such as a square, a circle, a rectangle, or a triangle. Hollow sections are usually extruded by placing a pin or mandrel inside of the die and in most cases positive pressure is applied to the internal cavities through the pin.

Coextrusion refers to the extrusion of multiple layers of material simultaneously. This type of extrusion utilizes two or more extruders to melt and deliver a steady volumetric throughput of different molten plastics to a single extrusion head which combines the materials in the desired shape. The layer thicknesses are controlled by the relative speeds and sizes of the individual extruders delivering the materials.

The present catheter may be produced by coextrusion wherein an inner and outer layer of clear, molten polymer material is extruded through an inner and outer extruder to a single, round extrusion head. A pin may also be centered inside the inner extruder to provide a lumen 28 for the catheter 14. A middle extruder may also be positioned between the inner and outer extruder whereby a molten, radiopaque material may be extruded and embedded between the inner and outer layers. The middle extruder may also be rotatably fixed between the inner and outer extruder. As such, the middle extruder may rotate independent of the inner and outer extruders thereby permitting the material 40 to be embedded between the inner and outer layers in a helical configuration. The middle extruder may include multiple outlets whereby a helical formation of the embedded material may include multiple strands of extruded radiopaque material.

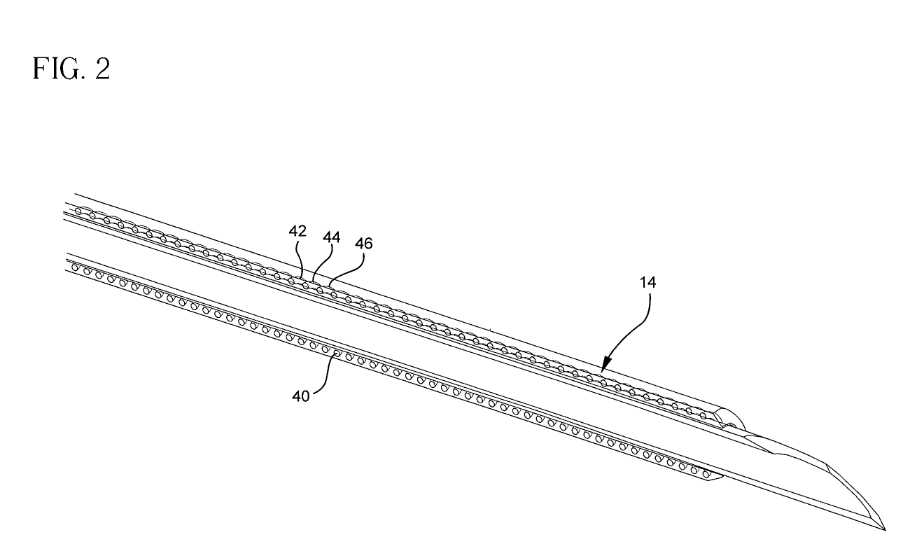

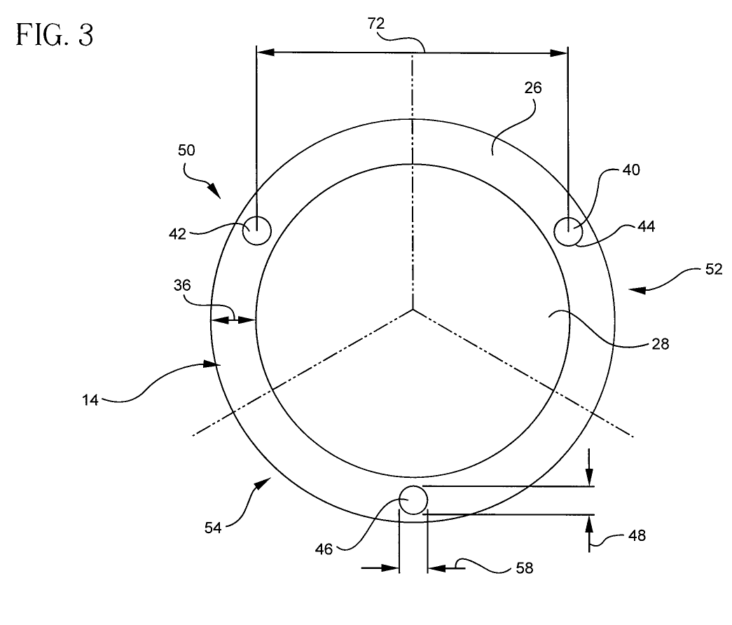

Referring now to FIGS. 2 and 3, a cross-section of the catheter of FIG. 1 is illustrated. The catheter 14, as illustrated, comprises a catheter wall 26 and three embedded strands 42, 44, and 46 of radiopaque material 40. The height 48 and cross-sectional shape of the material 40 may vary depending upon the needs of the catheter assembly 10. As previously discussed, the catheter 14 and the embedded material 40 may be configured during the extrusion process.

The height 48 of the material 40 is largely limited by the thickness 36 of the catheter wall 26. The height 48 and the width 58 of the material 40 may also be adjusted to achieve a desired flexibility or rigidity of the catheter 14. An embedded radiopaque material 40 is generally more rigid than the clear, flexible polymer material of the catheter 14. Therefore, the amount of radiopaque material embedded within the wall 26 of the catheter 14 will affect the flexibility of the catheter 14. For example, a catheter 14 with one embedded strand of radiopaque material 40 will be more flexible than a catheter with three embedded strands of radiopaque material 40, where the embedded strands of the two catheters are equal in height 48 and width 58. Therefore, the flexibility of a catheter 14 may also be affected by adjusting the height 48 and width 58 of the radiopaque material. The flexibility of the catheter may also be affected by intrinsic flexibility of the embedded material 40.

The cross-sectional shape of the material 40 may also be modified to accommodate a need of the catheter 14. For example, a cross-sectional shape may be selected to increase the radiopaque coverage of the material 40. A wide variety of profile shapes for the embedded material may be selected by configuring the one or more outlets of the middle extruder for the desired profile shape. In this way, the embedded material may be adjusted to comprise any desired height 48 and width 58. Conversely, the clear area of the catheter 14 (i.e. the area of the catheter 14 not comprising embedded material 40) may be increased or decreased by selecting a desired height 48 and width 58 of the embedded material 40.

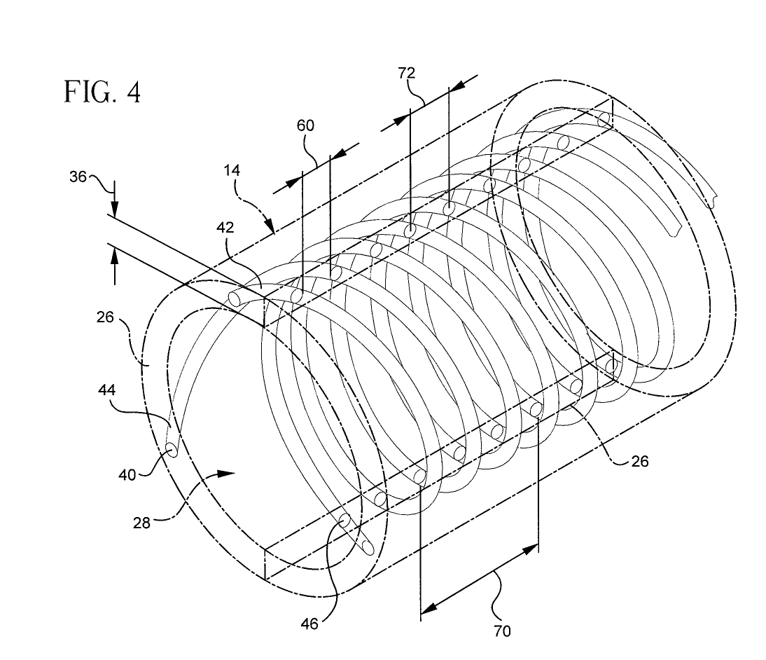

Referring now to FIG. 4, a portion of the catheter 14 is illustrated. The catheter 14 may include one or more strands of radiopaque material 40. In one embodiment the catheter 14 includes three (3) strands 42, 44, and 46 of radiopaque material 40. A first strand 42 may be embedded generally within a first quadrant 50 (illustrated in FIG. 3) of the catheter 14. The pitch 70 of the first strand 42 may be selected based on several factors. For example, where the catheter 14 includes a plurality of strands, the pitch 70 of each strand must be selected to provide sufficient room for the additional strands. Pitch 70 is defined as the distance from center to center of the adjacent coils of strands as embedded within the wall 26 of the catheter 14, and is expressed in coils per centimeter. Additionally, the pitch 70 may be selected to provide a sufficient window 60 between the adjacent coils of the radiopaque material 40. A window 60 is desirable as a source for visually observing the flow of a fluid through the catheter 14.

A second strand 44 and a third 46 strand may be embedded within a second 52 and third 54 quadrant (as illustrated in FIG. 3), respectively. The cumulative pitch 72 of the first, second and third 42, 44, and 46 strands may be selected based on several factors. The cumulative pitch 72 is defined as the distance from center to center of the adjacent coils of the several strands as embedded within the wall 26 of the catheter 14, and is expressed in coils per centimeter. In selecting the cumulative pitch 72, factors to consider may include the desired rigidity or flexibility of the catheter 14. Where a flexible catheter 14 is desired, a higher pitch, or a lower number of coils per centimeter may be selected. Conversely, where a more rigid catheter 14 is desired, a lower pitch, or higher number of coils per centimeter may be selected.

Additional factors may include the radiopacity of the catheter 14 as well as the transparency of the catheter 14. As previously discussed, the radiopacity of the catheter 14 may be increased or decreased by several factors. These factors include the number of individual strands of radiopaque, material 40, as well as the width 58, height 48, and cross-sectional shape of the individual strands. Additionally, the radiopacity of the catheter 14 may be increased by decreasing the cumulative pitch 72 or increasing the coils per centimeter. A decreased cumulative pitch 72 will increase the number of coils per centimeter thereby reducing the clear area of catheter, as defined above. Conversely, the radiopacity of the catheter 14 may be decreased by increasing the cumulative pitch 70 or decreasing the coils per centimeter. As visual observance of the fluid through the catheter 14 is desirable, considerations should be made to ensure an adequate window 60 for the catheter 14.

When exposed to X-ray film, the helical configuration of the radiopaque, material 40 results in a radiograph image that is unique from other physiological structures of the patient's body. Specifically, a radiograph of the catheter 14 reveals a distinctive, crisscrossed pattern that is not naturally replicated within the patient. As such, the catheter 14 may be easily located and distinguished from the patient's tissue via X-ray imaging. Additionally, other non-physiological patterns may be used for the embedded radiopaque material 40. For example, a zigzagged pattern or a wavy lined pattern may be used in place of the crisscrossed pattern. Additional non-physiological pattern are also anticipated and may be used within the scope of this invention.

Enhanced visibility of the catheter 14 may be beneficial in several ways. For example, X-ray imaging of a radiopaque catheter 14 may be used to track and assist a physician in the placement of a catheter for an angiogram or similar procedure. An angiogram is an imaging test that uses x-rays to view your body's blood vessels. Physicians often use this test to study narrow, blocked, enlarged, or malformed arteries or veins in many parts of your body, including your brain, heart, abdomen, and legs. The angiogram catheter is typically inserted at a location remote from the vein or veins of interest and then guided to the vein of interest by a physician. A catheter 14 embedded with a radiopaque material 40 may be useful in assisting the physician to visual the placement of the catheter using real-time x-ray equipment. Additionally, where the catheter has been inadvertently severed and released into the vasculature of a patient, the catheter 14 may be located via X-ray imaging, safely immobilized, and removed from the patient.

In addition to heightened visibility, the embedded material 40 may provide several additional benefits to the catheter 14. For example, the embedded material 40 may increase the strength of the catheter wall 26. Increased tube wall 26 strength may be desirable for infusion procedures requiring rapid infusion at high pressures, such as those involving severe hemorrhages or other hypovolemic conditions. Additionally, the increased tube wall 26 strength may prevent the catheter 14 from becoming kinked or partially occluded while inserted in the patient.

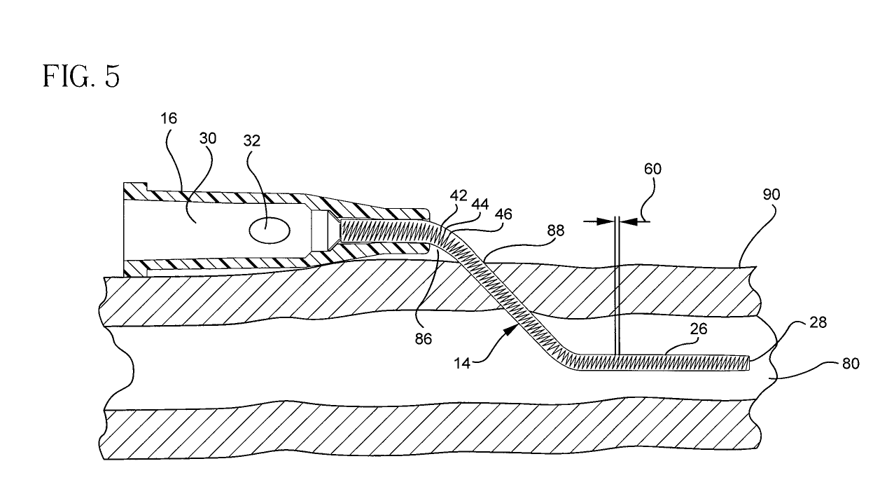

Referring now to FIG. 5, a catheter 14 is illustrated as inserted into the vasculature 80 of a patient 90. As compared to traditional radiopaque catheters having multiple, linear strands of embedded radiopaque material, the three embedded helical strands 42, 44, and 46 provide a flexible catheter 14 with optimal radiopaque detection. As previously discussed, the radiopaque material of traditional radiopaque catheters is added either evenly, resulting in uniformly opaque catheters, or is distributed in linear stripes leaving clear areas between the stripes for viewing fluid flow. In either configuration, the resultant radiograph of the opaque catheters yields linear patterns similar to the radiograph patterns of other surrounding tissues. Bones, muscles, ligaments, veins and other tissues are all generally linear and therefore display linear patterns when exposed to x-ray film. The non-physiological pattern of the helically embedded radiopaque material 40 provides a distinct pattern that may be easy detected and distinguished from surrounding tissue. In the event that the inserted catheter 14 becomes detached from the catheter adapter 16, the helical configuration of the embedded material 40 may be easily detected via x-ray, and the severed catheter safely removed.

The embedded radiopaque strands 42, 44, and 46 may strengthen the wall 26 of the catheter 14 such that the catheter 14 may bend between the catheter adapter 16 and the patient without forming a kink or an occlusion. A root region 86 of the catheter 14 is generally required to bend in order to accommodate a transition of the catheter 14 from the catheter adapter 16 to the insertion site 88 on the patient 90. The root region 86 of the catheter 14 may be kinked or occluded if the catheter 14 is over-inserted into the vasculature 80 of the patient 90. The embedded radiopaque strands 42, 44, and 46 may strengthen the wall 26 of the catheter 14 thereby preventing a kink or occlusion from forming at the root region 86. As such, a fluid may flow through the lumen 28 of the catheter 14 without disturbance.

Traditional radiopaque catheters are generally less flexible than radiopaque catheters implementing a helical configuration of embedded radiopaque material. To achieve a sufficient level of x-ray detection, traditional radiopaque catheters required either uniform coverage or multiple, linear strands of embedded radiopaque material. A uniformly embedded catheter is undesirable due to the decreased flexibility of the catheter, as well as the resultant opacity of the catheter. The flexibility of the traditional catheter, as well as the ability to view the flow of a fluid through the catheter, has been improved by embedding linear strands of radiopaque material along the length of the catheter.

Traditional radiopaque catheters have included six embedded strands of radiopaque material. As such, sufficient observation windows are provided between the stripes of embedded material, and yet the embedded stripes provide sufficient radiopaque detection via x-ray. Although more flexible than the uniformly embedded catheter, the six strands of embedded material still result in a semi-flexible catheter. The helically configured material 40 of the current radiopaque catheter 14 provides increased flexibility and visibility for a user. For example, the helical configuration of the radiopaque material 40 provides increased coverage with fewer strands of material 40. Therefore, the present catheter 14 may incorporate fewer strands of radiopaque material 40 and achieve greater coverage and detection than traditional radiopaque catheters.

Less embedded radiopaque material 40 will provide a more flexible catheter. As previously discussed, radiopaque materials 40 are typically less flexible than the clear polymer material of the catheter 14. As such, the flexibility of the catheter 14 may be increased by limiting the overall amount of radiopaque material 40 embedded in the catheter 14. By embedding the radiopaque material 40 in a helical configuration, the embedded material 40 provides increased coverage and therefore less material 40 is required to achieve a sufficient level of detection via x-ray. For example, in one embodiment three strands of embedded material 40 provides sufficient detection via x-ray when the material 40 is embedded in a helical configuration.

In addition to improved flexibility, increased wall 26 strength, and optimal radiopaque detection, the helical configuration provides a sufficient window 60 for observing the flow of a fluid through the catheter 14. A window 60 is defined as the clear portions of the catheter 14 not embedded with a radiopaque material 40. The windows 60 allow a user to visualize a flow of a fluid through the catheter 14. A window 60 is desirable for many reasons.

For example, when inserting a catheter 14 into a patient 90, a user may desire to visualize a flashback of the patient's 90 blood through the catheter 14 to verify that the catheter 14 is properly positioned within the patient's 90 vasculature 80. A window 60 allows a user to visualize a flashback within the catheter 14. A window 60 may also be desirable to visualize the flow of a fluid through the catheter 14. In the event that the vein of the patient 90 collapses or fails, the flow through the catheter 14 will cease. A window 60 allows a user to visualize the flow of a fluid through the catheter 14 and detect a failure of the patient's 90 vein.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A vascular catheter, comprising:
a tubular member; and
a radiopaque material attached to or disposed within the tubular member in a wound helical pattern which, when viewed by x-ray produces a non-physiological pattern.

2. The vascular catheter of claim 1, further comprising a catheter adapter coupled to an end of the tubular member.

3. The vascular catheter of claim 1, wherein the radiopaque material is embedded within a wall of the tubular member.

4. The vascular catheter of claim 3, wherein the radiopaque material extends the length of the tubular member.

5. The vascular catheter of claim 4, wherein the radiopaque material further comprises a plurality of helical strands.

6. The vascular catheter of claim 5, wherein the plurality of helically wound strands are embedded within the wall of the tubular member and circumscribes the length of the tubular member.

7. The vascular catheter of claim 6, wherein the radiopaque material supports a portion of the tubular member.

8. A method for optimizing the detection of a vascular catheter via x-ray detection; comprising:
providing a tubular member having an outer surface, an inner surface and a middle portion interposed between the inner and outer surfaces; and
coextruding a radiopaque material within the middle portion of the tubular member in a pattern which, when viewed by x-ray produces a non-physiological pattern;
wherein the tubular member is a vascular catheter.

9. The method of claim 8, further comprising the step of attaching a first end of the tubular member to a catheter adapter.

10. The method of claim 9, wherein the radiopaque material extends the length of the tubular member.

11. The method of claim 10, wherein the radiopaque material further comprises a plurality of strands wound in a helical pattern.

12. The method of claim 11, wherein the plurality of strands is embedded within the wall of the tubular member and circumscribes the length of the tubular member.

13. The method of claim 12, wherein the helical strands support a portion of the tubular member.

14. A vascular catheter detectible by x-ray; comprising:
a tubular member having an outer surface, an inner surface and a middle portion interposed between the inner and outer surfaces; and
at least one strand of a radiopaque material coextruded within the middle portion of the tubular member;
wherein the radiopaque material displays a non-physiological pattern when exposed to x-ray film.

15. The vascular catheter of claim 14, further comprising a catheter adapter attached to an end of the tubular member.

16. The vascular catheter of claim 14, wherein the radiopaque material comprises a plurality of extruded strands in a wound helical pattern.

17. The vascular catheter of claim 16, wherein the plurality of coextruded strands is embedded within the middle portion of the tubular member such that no coextruded strand crosses over another coextruded strand.

18. The vascular catheter of claim 17, wherein the radiopaque material supports a portion of the tubular member.

19. The vascular catheter of claim 18, wherein the radiopaque material prevents the tubular member becoming kinked.

20. The vascular catheter of claim 14, wherein the placement of the radiopaque material provides at least one window through which a fluid within the tubular member can be observed.
